(19) United States
(12) Patent Application Publication
(10) Pub. No.: US 2006/0210501 A1
(43) Pub. Date: Sep. 21, 2006

(54) Color cosmetic comprising Yerba Santa extract

(75) Inventors: Charles Benard, Gravih Bay, CA (US); Marc Cawdrey, Tiburon, CA (US); Esmeralda Mancia, (US); Laura DuPriest, Loomis, CA (US)

Correspondence Address: MATHEW J. TEMMERMAN, 423 E. STREET, DAVIS, CA, 95616, US

(73) Assignee: Laura DuPriest LLC

(21) Appl. No.: 11/359,186

(22) Filed: Feb. 22, 2006

Related U.S. Application Data
(60) Provisional application No. 60/655,291, filed on Feb. 22, 2005.

(51) Int. Cl. A61K 8/97; A61K 8/26; A61K 8/25

(52) U.S. Cl. 424/63; 424/74

(57) ABSTRACT

The application discloses and claims a color cosmetic composition providing moisturization, hydration, and lubrication through the use of Yerba Santa fluid extract. Specific formulations for anti shine pressed powder, blush, brow wax, concealer, eye shadow, lipstick and gloss, mascara, shimmer shadow and foundation compositions are disclosed.

### RELATED APPLICATION

This application claims priority from the provisional application with Ser. No. 60/655,291, which was filed on Feb. 22, 2005. The disclosure of that provisional application is incorporated herein by reference as if set out in full.

### BACKGROUND OF THE INVENTION

1. Field of the Invention

The present application relates to color cosmetic (makeup) compositions, specifically those having an application to the integumentary system, including the skin, lips, eyelashes, eyebrows and hair.

2. General Background

Cosmetics are substances used for the purposes of cleansing and beatifying the human body. Color cosmetics are a specific subset of cosmetics and are commonly referred to as “makeup”. The use of makeup is pervasive throughout society for millennia; the first evidence dating to ancient Egypt.

There are many types of makeup currently available, including lip color to color the lips, blush used to color the cheeks and emphasize cheekbones, mascara to enhance the eyelashes, eyeliner and eye shadow to color and emphasize the eyelids, foundation, usually a cream or powder (but also available as a stick) to even the skin tone of the face, brow wax to shape the eyebrows, and shimmer shadow, a shimmer cream eye shadow to darken or highlight the area around the eyes. Generally, makeup can be powders, dispersions, liquid or cream emulsions, and anhydrous creams or sticks.

There is growing demand in the markeplace for makeup that delivers some of the benefits normally associated with skin care products, benefits such as water solubility and a higher concentration of natural ingredients. One such effect highly sought after is moisturization, although as will be shown imparting moisturizing qualities into makeup has not proven to be a simple task. In general, moisturizing compounds and compositions function by increasing hydration of the corneal stratum water-absorbing cells, making the skin softer and more pliable. Moisturizers may also be used to repair dry or scaly skin, as well as skin damage caused by acne, aging, or external factors.

Typical commercial skin moisturizers include artificial and natural oils, humectants, emollients, lubricants, and naturally occurring skin lipids and sterols. Due to the near universal usage and vast market for moisturizing products, many advancements have been made. Commercial moisturizing compositions are complex and varied and not all have scientific evidence substantiating claims of increased moisturization.

In the past, increasing the moisturizing qualities of makeup has been difficult because to do so would generally diminish the other characteristics of the makeup.

For instance, liquid lip products are preferred to have a high gloss, yet to achieve high moisturization using conventional moisturizing compounds would require so much of said conventional moisturizing compounds that a high level of gloss could not be achieved. As an example, typical aloe vera moisturizers comprise between 20% and 80% aloe vera. Because such a large amount of aloe vera is needed to achieve a significant moisturizing effect, it cannot practically be added to liquid lip products.

The difficulties in introducing a moisturizing quality into makeup extend beyond just liquid lip products. For instance, in eye shadow, blush, or foundation, so much aloe vera would need to be added, that said makeup products would no longer exhibit their traditional qualities as makeup.

### DESCRIPTION OF THE PRIOR ART

An additional source of moisturizing compounds can be found in fluid extract obtained from Yerba Santa, an evergreen aromatic shrubby plant native to the western and southwestern parts of North America and northern Mexico.

The primary constituents in Yerba Santa are five phenolic bodies, eriodictyol, homoeriodictyol, chrysocriol, zanthoeridol and eridonel, and it further comprises acetic acid, butyric acid, cerotic acid, chrysoeriodictyol, chrysin, chrysoeriol, cirsimaritin, eriodictyol, eriodictyonic acid, eriodictyonine, eriodonol, fats, formic acid, gums, hispidulin, homoeriodictyol, naringenin, pentatriacontaine, phytosterols, pinocembrin, sakuranetin, sugars, tannin, triacontane, xanthoeriodictyol, and trace volatile oil.

Yerba Santa was used medicinally for centuries by Native Americans and to some extent California missionaries. It was boiled to make tea for coughs, cold, asthma, and other respiratory ailment. Such use died out in the late 1800s. The medicinal use of Yerba Santa was “rediscovered” in the early 1990s as part of a treatment for throat-cancer patients recovering through chemotherapy, and as part of a medical products regime for transdermal drug delivery and vaginal dryness.

The fluid extract from the Yerba Santa plant is commonly used in combination with bitter substances in order to mask their bitter taste. However, the extract also comprises mucopolysaccharides that increase the hydration and lubricity in the skin, even more so than the better-known Aloe vera. Like aloe vera, Yerba Santa achieves its moisturizing effect via hydrogen bonding of water by its polysaccharide components. The primary difference between Yerba Santa extract and aloe vera is that far less Yerba Santa extract is needed to achieve the same moisturizing effect. Whereas typical concentrations of aloe vera in a composition are, as described previously, between 20% and 80%, Yerba Santa extract composition need not exceed even 5% to achieve a similar effect.

There are other benefits to Yerba Santa as well. The polysaccharide components of Yerba Santa exhibit mucilaginous properties; Yerba Santa is a natural anti inflammatory and comprises twelve flavonoids commonly known for their antioxidant activity. Combined, these moisturization and anti-inflammatory properties have prompted the extract to recently be developed for use skin care, promoting a vital, healthy looking and feeling skin.

U.S. Pat. Nos. 4,938,963 and 4,983,378 disclose a moisturizing effect of Yerba Santa extract according to a method of treating dry mouth syndrome, wherein an amount of eriodictyon fluid extract and sweetener is orally administered to an affected individual. The '963 patent discloses a composition to be orally ingested or chewed as a gum containing a range of between 0.25% to 10% by weight Yerba Santa extract.

U.S. Pat. No. 5,015,474 further discloses Yerba Santa extract as a vehicle through which to impart moisture to a dermal or mucosal membrane substrate. As with the '963 patent, described above, the composition disclosed contains a range of 0.25% to 10% by weight Yerba Santa extract. The '474 patent further discloses a nasal decongestant spray comprising between 1% and 10% Yerba Santa fluid extract. It is noted that the percentage of the composition comprising Yerba Santa extract is again relatively low.

U.S. Pat. No. 5,128,132 provides formulations for alleviating the dryness of mucosal membranes in the upper and lower respiratory tract and gastrointestinal tract. In this instance Yerba Santa extract is used either as an aerosol spray or as a delayed release tablet, in an amount by weight of up to 80% Yerba Santa extract.

U.S. Pat. No. 5,248,501 advanced the art by disclosing a composition including Yerba Santa extract as an excipient for the delivery of a drug to the skin or mucosa. One such disclosed method involves moisturizing and increasing the permeability of the skin through the use of compositions comprising Yerba Santa extract. The extract is disclosed to increase the permeability of the skin, thereby allowing therapeutic levels of drugs to pass through.

The above referenced publications show that the pharmaceutical uses of Yerba Santa extract are currently becoming known. The present invention advances the art by disclosing formulations that successfully incorporate Yerba Santa extract into various makeup products.

### SUMMARY OF THE INVENTION

The invention discloses and claims a makeup composition providing moisturization, hydration, and lubrication through the use of Yerba Santa fluid extract. Specifically, anti shine pressed powder, blush, brow wax, concealer, eye shadow, lipstick and gloss, mascara, shimmer shadow and foundation compositions are claimed.

It is a primary object of the present invention to provide makeup compositions and methods of creating and using such compositions, wherein said compositions comprise a specific amount of Yerba Santa extract.

It is an additional object of the invention to overcome the tendency of many makeup products and make-up products to contribute to skin dryness.

It is yet a further object of the invention to provide various makeup products with superior hydration as well as the ability to reduce the appearance of fine lines, wrinkles and imperfections.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### DETAILED DESCRIPTION

The invention discloses and claims a makeup composition providing moisturization, hydration, and lubrication through the use of Yerba Santa fluid extract. Specifically, anti shine pressed powder, blush, brow wax, concealer, eye shadow, lipstick and gloss, mascara, shimmer shadow and foundation compositions are claimed.

As used in this application, “Yerba Santa” extract refers generally to extract obtained from a plant of the genus Eriodictyon, which is itself a member of the waterleaf family. In a preferred embodiment of the invention, the extract is obtained from Eriodictyon californicum, a specific species within the Eriodictyon genus.

Depending on the makeup to which it is added, Yerba Santa extract may affect the viscosity, or may require the addition of specific binders in order to maintain the general characteristics of said makeup. In some cases, the amount of other ingredients need to be reduced. The current application thus discloses specific formulations through testing have been shown to provide maximum moisturization while still maintaining the cosmetic's original qualities. This invention is described through a series of specific embodiments that are presented in the “Examples” portion of this application. For each exemplary embodiment, constituents with percentage weights are provided and a manufacturing process follows.

The general benefits of Yerba Santa are known in the art have been described in a previous section of this application. The primary benefit is that moisturizing and lubricating effects have been shown even when the extract is used in relatively small concentrations, (0.25% to 5%) compared to, for example, a popular moisturizer, aloe vera which in turn requires 20% to 80% concentrations to achieve a notable difference. This powerful moisturization and lubricity performance makes Yerba Santa particularly effective as an ingredient in color cosmetics and make-up where the benefits of moisturization and lubricity can be achieved while maintaining customary product integrity.

For example, a small amount of Yerba Santa, when properly formulated into a lip color cosmetic (i.e. lipstick, lip stain or lip gloss) adds a high degree of moisturization and lubricity performance without changing the basic characteristics of the product. If this same effect were to be brought about through the addition of aloe vera to such lip products, the amount of aloe vera that would need to be added would negate the other qualities of the product, and would give the product a gelatinous appearance. In other words, moisturizing qualities added to lip products allow for longer-lasting lip colors, may be applied without dragging, and help moisturize the lips rather than contribute to drying or chapping.

Specific to lip stains, the addition of Yerba Santa extract has shown to relieve the dryness that can be caused over time. Normally a lip stain offers long lasting effects but along with these long lasting effects normally come negative side effects such as dry chapped lips. By including Yerba Santa extract, lip stains may be used daily without the aforementioned negative side effects.

In a pressed anti-shine powder, Yerba Santa adds a silky feel and applies smoothly without the powdery feel and results of other products. The effects of Yerba Santa are similar in mascara, eye shadows, blushes, liners, foundation sticks and other make-up products.

By adding Yerba Santa to powdered pigments, the powders adhere readily to the skin because of the moisture base. Because of this, less powder is needed to provide the same amount of color. Tests have also shown that the wearing time of powders and pigments are increased.

When used with mascara, Yerba Santa creates smoother lashes and conditions the lashes increasing their strength and decreasing the likelihood of bending or breaking when using a lash curler. Traditionally, mascaras have required a quick drying agent, but this quick drying agent causes brittle lashes, and a grainy, gritty outward appearance. Here, the components shown in the ‘Example’ portion of this application were added together in the amount shown therein.

When Yerba Santa extract is used in foundations, similar hydrating and moisturizing effects were found. To achieve similar levels of hydration and moisturization using aloe vera would fundamentally change the composition of the foundations so that they were no longer suitable for their intended purpose. Here, the components shown in the ‘Example’ portion of this application were added together in the amount shown therein. The mixture described below achieves a composition that exhibits a unique silkiness to the touch, provides smooth consistent coverage upon application, that facilitates loading of the foundation applicator (including but not limited to a sponge, brush, puff, or an extruded stick similar to lipstick) and finally that provides a strong moisturizing effect. Through the applicant's novel formulas, foundation that in the past has been notoriously heavy and drying is now light and hydrating.

When used in blushes, similar hydrating and moisturizing effects were found. To achieve similar levels of hydration and moisturization using Aloe vera would fundamentally change the composition of the blush so that it was no longer suitable for its intended purpose.

When used in an eye shadow, the shadow can be applied in one stroke instead of multiple strokes.

Benefits similar to the above were found when Yerba Santa extract was formulated into various other makeup products. Additionally, it causes many of said products to feel light on the skin and provides coloring that does not appear washed out.

### EXAMPLES

One skilled in the art will appreciate that the invention disclosed herein may be practiced in the absence of any component which has not been specifically disclosed. The following examples are provided as further illustration of the invention and are not to be construed as limiting the scope of what the inventors regard as their inventions. Efforts have been made to insure accuracy with respect to numbers used (e.g., amounts, temperature, etc.) but some experimental errors and deviation should be accounted for. Unless stated to the contrary all parts and percentages are expressed on a weight basis, temperature is in degrees C, and pressure is at or near atmospheric.

Many of the following examples comprise talc, a mineral having a formula of either H₂Mg₃(SiO₃)₄ or Mg₃Si₄O₁₀(OH)₂. Talc is a metamorphic mineral having a hardness of 1, and may exist in powder form known as talcum powder.

Many of the following examples comprise iron oxide, which may include FeO, Fe₂O₃ and Fe₃O₄.

Many of the following examples comprise paraffin, a high molecular weight alkane hydrocarbon having the general formula Cₙ,H₂ₙ₊₂, where n is greater than about 20. It is mostly found as a white, odorless, tasteless, waxy solid, with a typical melting point between about 47° C. and 65° C.

Many of the following examples comprise ozokerite, a naturally occurring odouriferous mineral wax generally comprising a mixture of various hrydrocarbons.

Many of the following examples comprise Titanium dioxide, the naturally occuring oxide of titanium, which when used as a pigment, is referred to as titanium white or Pigment White 6 and has a very high refractive index (n=2.4).

The following examples may comprise castor oil, a vegetable oil obtained from the castor seed (Ricinus communis).

The following examples were formulated through testing and a great deal of experimentation and adjustment was necessary. The inventors found that when adding Yerba Santa extract to existing makeup compositions, the makeup did not retain many of the original characteristics that made the makeup desirable in the first place. Through calculating and adjusting the amounts of other ingredients, or adding more or different types of binders, the inventors created-various makeup compositions that provided all the benefits of Yerba Santa extract, while still retaining and in some cases enhancing their original characteristics. These optimal formulations are provided below.

ANTI SHINE NEUTRAL FACE POWDER/FINISHING POWDER

| COMPONENT | PERCENTAGE |
|---|---|
| MICA (CI 77019) | 69.3100 |
| TALC | 21.4700 |
| ETHYLHEXYL PALMITATE | 3.3000 |
| ZINC STEARATE | 2.9500 |
| GLYCINE SOJA (SOYBEAN) OIL | 1.5000 |
| ERIODICTYON CALIFORNICUM LEAF EXTRACT | 0.5000 |
| IRON OXIDES (CI 77492, CI 77492, CI 77499) | 0.3800 |
| METHYLPARABEN | 0.2900 |
| PROPYLPARABEN | 0.2000 |
| ETHYLPARABEN | 0.1000 |
| | 100.0000 |

MANUFACTURING PROCEDURES
ANTI-SHINE FACE POWDER/FINISHING POWDER

| PHASE | |
|---|---|
| A | MICA |
| A | TALC |
| A | ETHYLHEXYL PALMITATE |
| A | ZINC STEARATE |
| B | GLYCINE SOJA (SOYBEAN) OIL |
| B | ERIODICTYON CALIFORNICUM LEAF EXTRACT |
| C | METHYLPARABEN |
| C | PROPYLPARABEN |
| C | ETHYLPARABEN |
| D | IRON OXIDES (CI 77491, CI77492, CI77499) |
| D | TITANIUM DIOXIDE (CI77891) |

Blend all phase A ingredients in a clean sanitized stainless steel vessel with high speed intensification bar. Next add phase B ingredients to phase A while product is being mixed in the blender. Next add phase C ingredients to the phase A and B ingredients one at a time. Next add phase D colors, blend all pigments with phase A, B, and C. intensify until pigments are dispersed and no colors are seen or draw down.

BLUSH - ALL SHADES

CONTAINS
TALC
ETHYLHEXYL PALMITATE
ZINC STEARATE
GLYCINE SOJA (SOYBEAN) OIL
ERIODICTYON CALIFORNICUM LEAF EXTRACT
METHYLPARABEN
PROPYLPARABEN
ETHYLPARABEN
MAY CONTAIN
TITANIUM DIOXIDE (CI 77891)
IRON OXIDES (CI 77491, CI 77492, CI 77499)
CARMINE (CI 75470)
MICA (CI 77019)
RED 30 (CI 73360)

BLUSH FACE KIT - LIGHT PEACH

| COMPONENT | PERCENTAGE |
|---|---|
| MICA (CI 77019) | 50.7798 |
| TALC | 34.6400 |
| ETHYLHEXYL PALMITATE | 3.5900 |
| ZINC STEARATE | 3.0000 |
| TITANIUM DIOXIDE (CI 77891) | 2.4702 |
| IRON OXIDES (CI 77491, CI 77492, CI 77499) | 1.5900 |
| GLYCINE SOJA (SOYBEAN) OIL | 1.5000 |
| ERIODICTYON CALIFORNICUM LEAF EXTRACT | 0.5000 |
| RED 30 (CI 73360) | 1.0000 |
| CARMINE (CI 75470) | 0.3300 |
| METHYLPARABEN | 0.3000 |
| PROPYLPARABEN | 0.2000 |
| ETHYLPARABEN | 0.1000 |
| | 100.00000 |

BLUSH - MAUVE

| COMPONENTS | PERCENTAGE |
|---|---|
| MICA (CI 77019) | 58.08920 |
| TALC | 11.59000 |
| IRON OXIDES (CI 77491, CI 77492, CI 77499) | 6.58500 |
| TITANIUM DIOXIDE (CI 77891) | 6.06580 |
| ETHYLHEXYL PALMITATE | 6.00000 |
| CARMINE (CI 75470) | 4.06000 |
| GLYCINE SOJA (SOYBEAN) OIL | 3.00000 |
| ZINC STEARATE | 3.00000 |
| ERIODICTYON CALIFORNICUM LEAF EXTRACT | 1.00000 |
| METHYLPARABEN | 0.30000 |
| PROPYLPARABEN | 0.21000 |
| ETHYLPARABEN | 0.10000 |
| | 100.00000 |

MANUFACTURING PROCEDURES - BLUSH ALL SHADES

| PHASE | |
|---|---|
| A | TALC |
| A | ETHYLHEXYL PALMITATE |
| A | ZINC STEARATE |
| B | GLYCINE SOJA (SOYBEAN) OIL |
| B | ERIODICTYON CALIFORNICUM LEAF EXTRACT |
| C | METHYLPARABEN |
| C | PROPYLPARABEN |
| C | ETHYLPARABEN |
| D | TITANIUM DIOXIDE (CI 77891) |
| D | IRON OXIDES (CI 77491, CI 77492, CI 77499) |
| D | CARMINE (CI 75470) |
| D | MICA (CI 77019) |
| D | RED 30 (CI 73360) |

Blend all phase A ingredients in a clean sanitized stainless steel vessel with high speed intensification bar. Then add phase B ingredients to phase A while product is being mixed in the blender. Next add phase C to phase A and B one at a time. Next add phase D colors, blend all pigments with phase A, B, and C ingredients. Intensify until pigments are dispersed and no colors are seen or drawn down.

BROW WAX/BROW POWDER

| COMPONENT | PERCENTAGE |
|---|---|
| OCTYL PALMITATE | 32.9536800 |
| ALUMINUM STARCH OCTENYLSUCCINATE | 20.0690880 |
| CAPRYLIC/CAPRIC TRIGLYCERIDE | 15.9721440 |
| OZOKERITE | 11.9840560 |
| BEESWAX (CERA ALBA) | 10.3809040 |
| EUPHORBIA CERIFERA (CANDELILLA) WAX | 5.3339440 |
| BORON NITRIDE | 1.7515920 |
| ERIODICTYON CALIFORNICUM LEAF EXTRACT | 0.2600000 |
| GLYCINE SOJA (SOYBEAN) OIL | 0.7800000 |
| RETINYL PALMITATE | 0.0494800 |
| TOCOPHERYL ACETATE | 0.0494800 |
| ASCORBIC ACID | 0.0494800 |
| METHYLPARABEN | 0.2474000 |
| PROPYLPARABEN | 0.1187520 |
| | 100.0000000 |

| | BROW WAX EYE KIT |
|---|---|
| PHASE | MANUFACTURING PROCEDURES |
| A | OCTYL PALMITATE |
| A | ALUMINUM STARCH OCTENYLSUCCINATE |
| A | CAPRYLIC/CAPRIC TRIGLYCERIDE |
| A | OZOKERITE |
| A | BEESWAX (CERA ALBA) |
| A | EUPHORBIA CERIFERA (CANDELILLA) WAX |
| A | BORON NITRIDE |
| B | ERIODICTYON CALIFORNICUM LEAF EXTRACT |
| B | GLYCINE SOJA (SOYBEAN) OIL |
| B | RETINYL PALMITATE |
| B | TOCOPHERYL ACETATE |
| B | ASCORBIC ACID |
| B | METHYLPARABEN |
| B | PROPYLPARABEN |

Preweigh, Phase A and heat it up to 80° C. Add Phase B and mix until batch is uniform.

TOUCH UP CONCEALER

| COMPONENT | PERCENTAGE |
|---|---|
| TITANIUM DIOXIDE (CI 77891) | 29.4500 |
| GLYCERYL TRIACETYL RICINOLEATE | 25.2000 |
| ISOPROPYL PALMITATE | 21.7000 |
| OZOKERITE | 4.9400 |
| TALC | 4.8600 |
| BEESWAX (CERA ALBA) | 4.6700 |
| IRON OXIDES (CI 77491, CI 77492, CI 77499) | 3.1700 |
| COPERNICIA CERIFERA (CARNAUBA) WAX | 1.8600 |
| EUPHORBIA CERIFERA (CANDELILLA) WAX | 1.8600 |
| GLYCINE SOJA (SOYBEAN) OIL | 1.5000 |
| ERIODICTYON CALIFORNICUM LEAF EXTRACT | 0.5000 |
| PROPYLPARABEN | 0.2900 |
| | 100.0000 |

| | TOUCH UP CONCEALER |
|---|---|
| PHASE | MANUFACTURING PROCEDURES |
| A | GLYCERYL TRIACETYL RICINOLEATE |
| A | ISOPROPYL PALMITATE |
| A | OZOKERITE |
| A | BEESWAX (CERA ALBA) |
| A | TALC |
| A | COPERNICIA CERIFERA (CARNAUBA) WAX |
| A | EUPHORBIA CERIFERA (CANDELILLA) WAX |
| A | ERIODICTYON CALIFORNICUM LEAF EXTRACT |
| | PROPYLPARABEN |
| B | TITANIUM DIOXIDE (CI 77891) |
| B | IRON OXIDES (CI 77491, CI77492, CI77489) |

Pre-weigh Phase A ingredients in a clean kettle and heat to 80-85° C. Mix until base is uniform. Take base and gradually add Phase B ingredients. Put the combination through a roller mill and mill until all pigment is dispersed, then add to Phase A.

EYE SHADOW - MID-BROWN

| COMPONENT | PERCENTAGE |
|---|---|
| MICA (CI 77019) | 60.8900 |
| TALC | 19.3200 |
| IRON OXIDES (CI 77491, CI 77492, CI 77499) | 6.6800 |
| TITANIUM DIOXIDE (CI 77891) | 3.4100 |
| ZINC STEARATE | 3.0500 |
| ETHYLHEXYL PALMITATE | 3.0500 |
| GLYCINE SOJA (SOYBEAN) OIL | 1.5000 |
| ERIODICTYON CALIFORNICUM LEAF EXTRACT | 0.5000 |
| CARMINE (CI 75470) | 1.0000 |
| METHYLPARABEN | 0.3000 |
| PROPYLPARABEN | 0.2000 |
| ETHYLPARABEN | 0.1000 |
| | 100.0000 |

EYE SHADOW - MERLOT

| COMPONENT | PERCENTAGE |
|---|---|
| MICA (CI 77019) | 44.3837 |
| IRON OXIDES (CI 77491, CI 77492, CI 77499) | 22.2588 |
| TALC | 14.2200 |
| TITANIUM DIOXIDE (CI 77891) | 8.1250 |
| ETHYLHEXYL PALMITATE | 3.2300 |
| ZINC STEARATE | 2.5900 |
| BIOTITE | 2.4100 |
| GLYCINE SOJA (SOYBEAN) OIL | 1.5000 |
| ERIODICTYON CALIFORNICUM LEAF EXTRACT | 0.5000 |
| CARMINE (CI 75470) | 0.3925 |
| METHYLPARABEN | 0.2000 |
| PROPYLPARABEN | 0.1300 |
| ETHYLPARABEN | 0.0600 |
| | 100.0000 |

EYE SHADOW - PINK CHAMPAGNE

| COMPONENT | PERCENTAGE |
|---|---|
| MICA (CI 77019) | 57.99950 |
| TALC | 23.63000 |
| TITANIUM DIOXIDE (CI 77891) | 5.23450 |
| IRON OXIDES (CI 77491, CI 77492, CI 77499) | 4.36600 |
| ZINC STEARATE | 3.00000 |
| ETHYLHEXYL PALMITATE | 3.00000 |
| GLYCINE SOJA (SOYBEAN) OIL | 1.50000 |
| ERIODICTYON CALIFORNICUM LEAF EXTRACT | 0.50000 |
| CARMINE (CI 75470) | 0.17000 |
| METHYLPARABEN | 0.30000 |
| PROPYLPARABEN | 0.20000 |
| ETHYLPARABEN | 0.10000 |
| | 100.00000 |

EYE SHADOW - BROWN

| COMPONENT | PERCENTAGE |
|---|---|
| MICA (CI 77019) | 65.0000 |
| IRON OXIDES (CI 77491, CI 77492, CI 77499) | 13.9100 |
| TALC | 7.0700 |
| TITANIUM DIOXIDE (CI 77891) | 6.4200 |
| ZINC STEARATE | 3.0000 |
| ETHYLHEXYL PALMITATE | 2.0000 |
| GLYCINE SOJA (SOYBEAN) OIL | 1.5000 |
| ERIODICTYON CALIFORNICUM LEAF EXTRACT | 0.5000 |
| METHYLPARABEN | 0.3000 |
| PROPYLPARABEN | 0.2000 |
| ETHYLPARABEN | 0.1000 |
| | 100.0000 |

EYE SHADOW - BEIGE FORMULATION 1

| CONTAINS | PERCENTAGE |
|---|---|
| MICA (CI 77019) | 79.2700 |
| TALC | 8.1500 |
| TITANIUM DIOXIDE (CI 77891) | 4.2300 |
| ZINC STEARATE | 3.0000 |
| ETHYLHEXYL PALMITATE | 2.0000 |
| GLYCINE SOJA (SOYBEAN) OIL | 1.5000 |
| ERIODICTYON CALIFORNICUM LEAF EXTRACT | 0.5000 |
| IRON OXIDES (CI 77491, CI 77492, CI 77499) | 0.7500 |
| METHYLPARABEN | 0.3000 |
| PROPYLPARABEN | 0.2000 |
| ETHYLPARABEN | 0.1000 |
| TOTAL | 100.0000 |

EYE SHADOW - DEEP PURPLE

| CONTAINS | PERCENTAGE |
|---|---|
| MANGANESE VIOLET (CI 77742) | 44.6000 |
| MICA (CI 77019) | 32.9600 |
| IRON OXIDES (CI 77491, CI 77492, CI 77499) | 8.8400 |
| ULTRAMARINES (CI 77007) | 4.1600 |
| ETHYLHEXYL PALMITATE | 3.0000 |
| ZINC STEARATE | 2.5100 |
| GLYCINE SOJA (SOYBEAN) OIL | 1.5000 |
| ERIODICTYON CALIFORNICUM LEAF EXTRACT | 0.5000 |
| TALC | 0.8400 |
| CARMINE (CI 75470) | 0.6000 |
| METHYLPARABEN | 0.2500 |
| PROPYLPARABEN | 0.1600 |
| ETHYLPARABEN | 0.0800 |
| | 100.0000 |

EYE SHADOW - MANUFACTURING PROCEDURES

| PHASE | |
|---|---|
| A | MICA (CI77019) |
| A | TALC |
| A | ZINC STEARATE |
| A | ETHYLHEXYL PALMITATE |
| B | GLYCINE SOJA (SOYBEAN) OIL |
| B | ERIODICTYON CALIFORNICUM LEAF EXTRACT |
| C | METHYLPARABEN |
| C | PROPYLPARABEN |
| C | ETHYLPARABEN |
| D | IRON OXIDES (CI 77491, CI77492, CI77499) |
| D | TITANIUM DIOXIDE (CI77891) |
| D | ULTRAMARINE BLUE (CI77007) |
| D | CHROMIUM OXIDE GREENS (CI77288) |
| D | YELLOW NO. 5 (CI 19140) |
| D | CARMINE (CI 75470) |
| D | MANGANESE VIOLET (CI77742) |

Blend all phase A ingredients in a clean sanitized stainless steel vessel with high sped intensification bar. Then add phase B to phase A while product is being mixed in a blender. Next add phase C to phase A and B, one at a time and combine with phases A, B, and C. Next add phase D colors, blend all pigments with phase A, B, and C. Intensify until pigments are dispersed and no colors are seen or draw down.

LIPGLOSS POT PINK - COPPER

| COMPONENT | PERCENTAGE |
|---|---|
| POLYBUTENE | 46.1820000 |
| MINERAL OIL (PARAFFINUM LIQUIDUM) | 21.5900850 |
| MICA (CI 77019) | 14.3087000 |
| OZOKERITE | 6.2345700 |
| IRON OXIDES (CI 77491, CI 77492, CI 77499) | 4.7240000 |
| TITANIUM DIOXIDE (CI 77891) | 2.9373000 |
| PARAFFIN | 2.8478900 |
| ERIODICTYON CALIFORNICUM LEAF EXTRACT | 0.2650000 |
| GLYCINE SOJA (SOYBEAN) OIL | 0.7950000 |
| ISOBUTYLPARABEN | 0.0346365 |
| | 100.0000000 |

LIPGLOSS POT PINK - PEACH

| CONTAINS | PERCENTAGE |
|---|---|
| POLYBUTENE | 52.956000 |
| MINERAL OIL (PARAFFINUM LIQUIDUM) | 24.756930 |
| OZOKERITE | 7.149060 |
| MICA (CI 77019) | 6.306800 |
| TITANIUM DIOXIDE (CI 77891) | 4.063100 |
| PARAFFIN | 3.265620 |
| ERIODICTYON CALIFORNICUM LEAF EXTRACT | 0.265000 |
| GLYCINE SOJA (SOYBEAN) OIL | 0.795000 |
| CARMINE (CI 75470) | 0.109600 |
| IRON OXIDES (CI 77491, CI 77492, CI 77499) | 0.130500 |
| ISOBUTYLPARABEN | 0.039717 |
| ISOPROPYLPARABEN | 0.052956 |
| BUTYLPARABEN | 0.039717 |
| RED 30 (CI 73360) | 0.070000 |
| TOTAL | 100.000000 |

LIPGLOSS POT PINK

| CONTAINS | PERCENTAGE |
|---|---|
| POLYBUTENE | 57.93600 |
| MINERAL OIL (PARAFFINUM LIQUIDUM) | 27.08508 |
| OZOKERITE | 7.82136 |
| PARAFFIN | 3.57272 |
| MICA (CI 77019) | 1.50620 |
| ERIODICTYON CALIFORNICUM LEAF EXTRACT | 0.26500 |
| GLYCINE SOJA (SOYBEAN) OIL | 0.79500 |
| CARMINE (CI 75470) | 0.01310 |
| TITANIUM DIOXIDE (CI 77891) | 0.80140 |
| IRON OXIDES (CI 77491, CI 77492, CI 77499) | 0.05930 |
| ISOBUTYLPARABEN | 0.04345 |
| ISOPROPYLPARABEN | 0.05794 |
| BUTYLPARABEN | 0.04345 |
| TOTAL | 100.00000 |

LIPSTICK - BERRY

| CONTAINS | PERCENTAGE |
|---|---|
| RICINUS COMMUNIS (CASTOR) SEED OIL | 48.212590 |
| GLYCERYL TRIACETYL RICINOLEATE | 7.285624 |
| IRON OXIDES (CI 77491, CI 77492, CI 77499) | 7.070000 |
| TITANIUM DIOXIDE (CI 77891) | 4.757000 |
| TALC | 4.395040 |
| BEESWAX (CERA ALBA) | 3.862564 |
| ISOPROPYL PALMITATE | 3.638586 |
| OZOKERITE | 3.380800 |
| HYDROGENATED LANOLIN | 3.372348 |
| MICA (CI 77019) | 3.168000 |
| ISOPROPYL MYRISTATE | 2.011576 |
| GLYCINE SOJA (SOYBEAN) OIL | 1.500000 |
| SQUALANE | 1.348094 |
| LANOLIN | 1.331190 |
| COPERNICIA CERIFERA (CARNAUBA) WAX | 1.267800 |
| EUPHORBIA CERIFERA (CANDELILLA) WAX | 1.267800 |
| ERIODICTYON CALIFORNICUM LEAF EXTRACT | 0.500000 |
| SILICA | 0.676160 |
| PROPYLPARABEN | 0.118328 |
| RED 7 (CI 15850) | 0.836500 |
| TOTAL | 100.000000 |

LIPSTICK - MICRO ANGEL

| CONTAINS | PERCENTAGE |
|---|---|
| RICINUS COMMUNIS (CASTOR) SEED OIL | 32.792430 |
| MICA (CI 77019) | 18.616800 |
| GLYCERYL TRIACETYL RICINOLEATE | 7.589048 |
| TITANIUM DIOXIDE (CI 77891) | 6.203200 |
| IRON OXIDES (CI 77491, CI 77492, CI 77499) | 4.852300 |
| TALC | 4.578080 |
| BEESWAX (CERA ALBA) | 4.023428 |
| OZOKERITE | 3.521600 |
| ISOPROPYL MYRISTATE | 3.790122 |
| HYDROGENATED LANOLIN | 3.512796 |
| ISOPROPYL PALMITATE | 2.095352 |
| GLYCINE SOJA (SOYBEAN) OIL | 1.500000 |
| SQUALANE | 1.404238 |
| LANOLIN | 1.386630 |
| COPERNICIA CERIFERA (CARNAUBA) WAX | 1.320600 |
| EUPHORBIA CERIFERA (CANDELILLA) WAX | 1.320600 |
| ERIODICTYON CALIFORNICUM LEAF EXTRACT | 0.500000 |
| CARMINE (CI 75470) | 0.165200 |
| SILICA | 0.704320 |
| PROPYLPARABEN | 0.123256 |
| | 100.000000 |

MANUFACTURING PROCEDURES - LIP STICK ALL SHADES

| PHASE | |
|---|---|
| A | POLYBUTENE |
| A | MINERAL OIL |
| A | OZOKERITE |
| A | PARAFFIN |
| B | ERIODICTYON CALIFORNICUM LEAF EXTRACT |
| B | GLYCINE SOJA (SOY BEAN) OIL |
| C | ISOBUTYLPARABEN |
| C | ISOPROPYLPARABEN |
| C | BUTYLPARABEN |
| D | MICA (CI77091) |
| D | TITANIUM DIOXIDE (CI77891) |
| D | IRON OXIDES (CI77492, CI77492, CI77499) |
| D | CARMINE (CI 75470) |
| D | MANGANESE VIOLET (CI7742 |
| D | RED#30 LAKE (CI73360) |
| D | RED #7 LAKE (CI 15850) |

Lip Glass Manufacturing Instructions: Preweigh phase A ingredients in a clean, sanitized kettle and heat to 80-85 degrees C. Once all raw materials are melted and dispersed, begin adding phase B ingredients to phase A one at a time while mixing. Next bring the batch to 70-75 degrees C and begin adding phase B ingredients while mixing. Once all raw materials are melted begin adding phase C ingredients while the product is being mixed. Once everything is uniform take some base in a separate kettle and mix with the phase D ingredients and put it through a roller mill. Mill until all pigments are dispersed then add it to phase A, B, and C ingredients while product is being mixed. Then cool batch to room temperature.

Lip Stick Manufacturing Instructions: Preweigh phase A in a clean sanitized kettle and heat to 80-85 degrees C. Once all raw materials are melted and dispersed begin adding phase B ingredients to phase A ingredients one at a time while mixing. Next bring the batch to 70-75 degrees C and begin adding phase B ingredients while mixing. Once all raw materials are melted begin to add phase C while the product is being mixed. Once everything is uniform take some base in a separate kettle and mix with phase D and put it through a roller mill and mill until all pigments are dispersed. Next add it to phase A, B, and C while product is being mixed. Finally cool the batch to room temperature.

MASCARA

| COMPONENTS | PERCENTAGE |
|---|---|
| WATER (AQUA) | 51.8600 |
| IRON OXIDES (CI 77419, CI 77492, CI 77499) | 17.7000 |
| SHELLAC WAX (SHELLAC CERA) | 9.2000 |
| GLYCERYL STEARATE | 4.3600 |
| BEESWAX (CERA ALBA) | 3.5400 |
| OZOKERITE | 2.4700 |
| SORBITAN SESQUIOLEATE | 2.3600 |
| STEARIC ACID | 2.3600 |
| TRIETHANOLAMINE | 1.7900 |
| GLYCINE SOJA (SOYBEAN) OIL | 1.5000 |
| PROPYLENE GLYCOL | 1.1800 |
| ERIODICTYON CALIFORNICUM LEAF EXTRACT | 0.5000 |
| PHENOXYETHANOL | 0.8496 |
| METHYLPARABEN | 0.1770 |
| ETHYLPARABEN | 0.0472 |
| BUTYLPARABEN | 0.0472 |
| PROPYLPARABEN | 0.0236 |
| ISOBUTYLPARABEN | 0.0354 |
| | 100.0000 |

Mascara Manufacturing Procedures

| PHASE | |
|---|---|
| A | WATER (AQUA) |
| B | SHELLAC WAX (SHELLAC CERA) |
| B | GLYCERYL STEARATE |
| B | BEESWAX (CERA ALBA) |
| B | OZOKERITE |
| B | SORBITAN SESQUIOLEATE |
| C | IRON OXIDES (CI77419, CI77492, CI77499) |
| D | TRIETHANOLAMINE |
| D | GLYCINE SOJA (SOYBEAN) OIL |
| D | PROPYLENE GLYCOL |
| D | ERIODICTYON CALIFORNICUM LEAF EXTRACT |
| E | STEARIC ACID |
| E | METHYLPARABEN |
| E | ETHYLPARABEN |
| E | BUTYLPARABEN |
| E | PROPYLPARABEN |
| E | ISOBUTYLPARABEN |
| F | PHENOXYETHANOL |

In a stainless steel kettle add Phase A water into main kettle and begin heating to 80-85° C. While heating add Phase B and mix until uniform. Add Phase C and begin homomixing until pigment is dispersed. Add Phase D ingredients one by one and continue mixing. Add Phase E ingredients one at a time. Once waxes seem to be softened, apply slow mixing. Add Phase F into kettle and continue mixing to complete batch.

SHIMMER EYE SHADOW - GOLD

| COMPONENTS | PERCENTAGE |
|---|---|
| WATER (AQUA) | 60.079477 |
| SYNTHETIC FLUORPHLOGOPITE | 17.910200 |
| TITANIUM DIOXIDE (CI 77891) | 6.706400 |
| MICA (CI 77019) | 4.845000 |
| SILICA | 2.882048 |
| BUTYLENE GLYCOL | 1.572656 |
| GLYCERIN | 1.385600 |
| TRIETHANOLAMINE | 1.385600 |
| ERIODICTYON CALIFORNICUM LEAF EXTRACT | 0.250000 |
| GLYCINE SOJA (SOYBEAN) OIL | 0.750000 |
| CARBOMER | 0.415680 |
| XANTHAN GUM | 0.173200 |
| PVP | 0.692800 |
| CITRIC ACID | 0.000139 |
| IRON OXIDES (CI 77492, CI 77492, CI 77499) | 0.258400 |
| PHENOXYETHANOL | 0.484960 |
| METHYLPARABEN | 0.207840 |
| | 100.0000000 |

SHIMMER EYE SHADOW GOLD
MANUFACTURING PROCEDURES

| PHASE | |
|---|---|
| A | WATER (AQUA) |
| B | SYNTHETIC FLUORPHLOGOPITE |
| B | SILICA |
| B | BUTYLENE GLYCOL |
| B | GLYCERIN |
| B | TRIETHANOLAMINE |
| B* | ERIODICTYON CALIFORNICUM LEAF EXTRACT |
| C | CARBOMER |
| C | XANTHAN GUM |
| C | PVP |
| D | CITRIC ACID |
| E | METHYLPARABEN |
| E | PHENOXYETHANOL |
| F | TITANIUM DIOXIDE (CI77891) |
| F | MICA (CI77019) |
| F | IRON OXIDES (CI77419, CI77492, CI77499) |

*contains 1% or less

Preweigh phase A in a stainless steel kettle add phase A and begin homomixing. Next add phase B ingredients one at a time and then add phase C and mix with the homomix until uniform. Next add phase D to phase A, B, and C and keep mixing. Add phase E ingredients one at a time. When phase A, B, C, D, E appears uniform, add phase F and mix until uniform.

FOUNDATION STICK/COMPACT/OTHER - MEDIUM

| COMPONENTS | PERCENTAGE |
|---|---|
| GLYCERYL TRIACETYL RICINOLEATE | 25.1067630 |
| ISOPROPYL PALMITATE | 21.8348130 |
| TITANIUM DIOXIDE (CI 77891) | 12.4000000 |
| SILICA | 10.3400000 |
| CYCLOPENTASILOXANE | 5.8168000 |
| OZOKERITE | 4.9806350 |
| TALC | 4.8861120 |
| BEESWAX (CERA ALBA) | 4.6970660 |
| IRON OXIDES (CI 77491, CI 77492, CI 77499) | 3.5500000 |
| COPERNICIA CERIFERA (CARNAUBA) WAX | 1.8759180 |
| EUPHORBIA CERIFERA (CANDELILLA) WAX | 1.8759180 |
| DIMETHICONE/VINYL | 1.4542000 |
| TRIMETHYLSILOXYSILICATE CROSSPOLYMER | |
| ERIODICTYON CALIFORNICUM LEAF EXTRACT | 0.2500000 |
| GLYCINE SOJA (SOYBEAN) OIL | 0.7500000 |
| PROPYLPARABEN | 0.1817750 |
| TOTAL | 100.0000000 |

| PHASE | MANUFACTURING PROCEDURES - FOUNDATION |
|---|---|
| A | GLYCERYL TRIACETYL RICINOLEATE |
| A | ISOPROPYL PALMITATE |
| A | SILICA |
| A | CYCLOPENTASILOXANE |
| A | OZOKERITE |
| A | TALC |
| A | BEESWAX (CERA ALBA) |
| A | EUPHORBIA CERIFERA (CANDELILLA) WAX |
| A | COPERNICIA CERIFERA (CARNAUBA) WAX |
| A | DIMETHICONE/VINYL TRIMETHYLSILOXYSILICATE |
| | CROSSPOLYMER |
| B | ERIODICTYON CALIFORNICUM LEAF EXTRACT |
| B | GLYCINE SOJA (SOYBEAN) OIL |
| B | PROPYLPARABEN |
| C | TITANIUM DIOXIDE (CI 77891) |
| C | IRON OXIDES (CI 77491, CI77492, CI77499) |

Manufacturing Procedures—Stick Foundation Medium: Preweigh Phase A in a clean kettle and start heat up to 60-65 degrees C. Take some base and add to Phase B. Put it through the roller mill and mill until all pigment is dispersed. Add to Phase A, then add Phase C and mix until uniform.

Manufacturing Procedures—Stick Foundation All shades: Preweigh Phase A in a clean kettle and heat it up to 80° C. While is been mix than bring the temperature at 60-65 c and add phase B and mixed until batch is uniform. Than take some base and add to Phase C and Put it through the roller mill and mill until all pigment is dispersed.

One skilled in the art will appreciate that the present invention can be practiced by other than the preferred embodiments, which are presented for purposes of illustration and not of limitation. In addition, further modifications may be made without departing from the true spirit and scope of the invention as defined and limited by the appended claims.

We claim:

1. A makeup composition comprising mica and Eriodictyon fluid extract.

2. The makeup composition according to claim 1, wherein the amount of Eriodictyon fluid extract is not more than 3% by weight relative to the total weight of the composition.

3. The makeup composition according to claim 1 wherein the amount of mica is not less than 40% by weight of the composition.

4. The makeup composition according to claim 1 further comprising talc, ethylhexyl palmitate, and zinc stearate.

5. The makeup composition according to claim 4, wherein the amount of talc is not less than 15%.

6. A makeup composition comprising a palmitate and Eriodictyon fluid extract.

7. The makeup composition according to claim 6 further comprising aluminum, caprylic/capric triglyceride, and a mineral wax.

8. The makeup composition according to claim 7, wherein said palmitate is octyl palmitate, wherein said aluminum is aluminum starch octenylsuccinate, and wherein said mineral wax comprises ozokerite.

9. The makeup composition according to claim 8, wherein the amount of octyl palmitate is not less than 25% by weight of the composition.

10. A makeup composition comprising titanium and Eriodictyon fluid extract.

11. The makeup composition according to claim 10, wherein said titanium is titanium dioxide.

12. The makeup composition according to claim 11 further comprising glyceryl triacetyl ricinoleate and isopropyl palmitate.

13. A makeup composition comprising Yerba Santa and a mineral that exhibits perfect basal cleavage.

14. The makeup composition of 13 wherein said mineral has a hardness of not greater than 1.

15. The makeup composition according to claim 13 wherein said makeup composition comprises an emulsion, a cream, a lotion, a solution, an anhydrous base, a gel, or an ointment.

16. The makeup composition according to 13 further comprising a pigment having a refractive index of n less than 2.4.

17. The makeup composition according to claim 16 wherein said makeup composition comprises an emulsion, a cream, a lotion, a solution, an anhydrous base, a gel, or an ointment.

18. The makeup composition according to claim 13 further comprising a second mineral that exhibits perfect basal cleavage, zinc, and Eriodictyon fluid extract.

19. The makeup composition according to claim 18 wherein said mineral that exhibits perfect basal cleavage and said second mineral that exhibits perfect basal cleavage comprise the minerals mica and talc.

20. The makeup composition according to claim 19 further comprising iron oxide and titanium oxide and wherein said zinc comprises zinc stearate.

21. The makeup composition according to claim 19 further comprising manganese violet or ultramarines.

22. A makeup composition comprising polybutene, mineral oil, a high molecular weight alkane hydrocarbon, a mineral wax, and Eriodictyon fluid extract.

23. The makeup composition according to claim 22 wherein said high molecular weight alkane hydrocarbon is paraffin, and wherein said mineral wax as ozokerite.

24. A makeup composition comprising castor oil, glyceryl triacetyl ricinoleate, and Eriodictyon fluid extract.

25. The makeup composition according to claim 24 further comprising mica and talc and iron oxide.

26. A makeup composition comprising iron oxide, a wax, glyceryl stearate, and Eriodictyon fluid extract.

27. The makeup composition according to claim 26 wherein said wax is shellac wax.

28. A makeup composition comprising a fluorphlogopite, silica and Eriodictyon fluid extract.

29. The makeup composition according to claim 28, further comprising titanium dioxide and mica.

30. A makeup composition comprising glyceryl triacetyl ricinoleate and Eriodictyon fluid extract.

31. The makeup composition according to claim 30 further comprising isopropyl palmitate, titanium dioxide, and silica.

32. The makeup composition according to claim 31 wherein the amount of glyceryl triacetyl ricinoleate is not less than 20% by weight of the composition, said isopropyl palmitate is not less than 15% by weight of the composition, and said titanium dioxide is not less than 10% by weight of the composition.

33. The makeup composition according to claim 32 wherein said composition is in the form of a powder.

34. The makeup composition according to claim 33, wherein said Eriodictyon fluid comprises fluid extract derived from Eriodictyon californicum.

35. The makeup composition according to claim 32 wherein said composition is in the form of an extruded stick.

36. The makeup composition according to claim 35, wherein said Eriodictyon fluid comprises fluid extract derived from Eriodictyon californicum.
